(19) United States
(12) Patent Application Publication
(10) Pub. No.: US 2024/0398505 A1
(43) Pub. Date: Dec. 5, 2024

(54) SURGICAL CUTTING TOOL AND METHODS FOR IDENTIFYING SAME

(71) Applicant: Medtronic PS Medical, Inc., Goleta, CA (US)

(72) Inventors: Michael Vu, Grand Prairie, TX (US); Aayush Malla, Fort Worth, TX (US); Haoran Li, Flower Mound, TX (US); John W. Kulas, Euless, TX (US)

(21) Appl. No.: 18/673,502

(22) Filed: May 24, 2024

Related U.S. Application Data
(60) Provisional application No. 63/469,621, filed on May 30, 2023.

(51) Int. Cl. A61B 90/90; A61B 17/00; A61B 17/16; A61B 90/00

(52) U.S. Cl. CPC A61B 90/90; A61B 17/1615; A61B 2017/00725; A61B 2017/00929; A61B 2090/0814

(57) ABSTRACT

A method for identifying a surgical tool for use with a surgical device includes integrating a variable indicator within a removably engageable component of a surgical tool of a surgical device. The method also includes engaging the surgical tool with the surgical device, wherein, upon engagement, the variable indicator aligns with one or more leads connected to a power console. The power control reads the variable indicator and identifies the surgical tool based on quantifying the variable indicator. The power control is then calibrated according to the operating parameters of the surgical tool stored within the power control.

### CROSS-REFERENCE TO RELATED APPLICATION

This application claims the benefit of and priority to U.S. Provisional Patent Application No. 63/469,621 filed May 30, 2023, the entire disclosure of which is incorporated by reference herein.

### FIELD

The present disclosure relates to surgical devices, systems, and methods and, more particularly, to surgical devices including systems for identifying, distinguishing, calibrating, and/or assessing amongst a variety of different surgical tools that are operably engageable with the surgical device.

### BACKGROUND

Powered surgical cutting devices and systems are utilized in a wide variety of surgical procedures to perform various different surgical cutting functions including, for example, drilling, tapping, resection, dissection, debridement, shaving, sawing, pulverizing, and/or shaping of anatomical tissue including bone.

Such powered surgical cutting devices and systems are precisely designed to ensure the devices function safely and effectively. Many powered surgical devices and systems have interchangeable tools of different sizes, with each tool designed to operate at different parameters. Accordingly, depending on the particular characteristics of the tool selected for use with the system, a clinician adjusts one or more setting on the system's surgical console for optimum tool usage. Authenticating a surgical tool, tracking the tool usage, or assessing the reliability of the surgical tool would be of value and allow clinicians to determine whether to continue or discontinue using a particular surgical tool during the course of a procedure.

### SUMMARY

As used herein, the term “distal” refers to the portion that is being described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. To the extent consistent, any of the aspects described herein may be used in conjunction with any or all of the other aspects described herein.

Provided in accordance with aspects of the present disclosure is a method for identifying a surgical tool for use with a surgical device which includes integrating a variable indicator within a removably engageable component of the surgical tool of the surgical device. The method also includes: engaging the surgical tool with the surgical device, wherein, upon engagement, the variable indicator aligns with one or more leads connected to a power console, e.g., an integrated power console (IPC); reading the variable indicator with the IPC and identifying the surgical tool based on quantifying the variable indicator; and calibrating the IPC according to the operating parameters of the surgical tool stored within the IPC.

In aspects according to the present disclosure, the variable indicator is a resistive indicator. In other aspects according to the present disclosure, the method further includes integrating the resistive indicator within a removeable shaft of a surgical cutting device. In yet other aspects according to the present disclosure, the method further includes aligning the resistive indicator between two bearings that support the removeable shaft, each bearing residing in registration with a corresponding lead connected to the IPC.

In aspects according to the present disclosure, the variable indicator is a resistive coating. In other aspects according to the present disclosure, the method further includes applying the resistive coating atop a removeable shaft of a surgical cutting device. In yet other aspects according to the present disclosure, the method further includes aligning the resistive coating between two bearings that support the removeable shaft, each bearing residing in registration with a corresponding lead connected to the IPC.

In aspects according to the present disclosure, the resistive coating is configured to wear during use of the surgical tool, effectively changing the electrical resistance of the surgical tool over time. In other aspects according to the present disclosure, the method further includes monitoring the condition of the surgical tool as a function of the wear of the resistive coating over time. In still other aspects according to the present disclosure, the method further includes layering the resistive coating to prevent reuse, wherein each layer includes a different quantifier. In yet other aspects according to the present disclosure, the method further includes removing the surgical tool from the surgical device when the IPC associates a thickness of the resistive coating to a condition of the surgical tool recommending replacement; and replacing the surgical tool according to the above method.

Provided in accordance with other embodiments of the present disclosure is a system for identifying a surgical tool for use with a surgical device which includes a selectively removable tool of the surgical device including an integrated variable indicator disposed therein. One or more leads are configured to align with the removable tool upon engagement with the surgical device, the one or more leads are connected to a power console, e.g., an integrated power console (IPC), configured to read the variable indicator and identify the surgical tool based on quantifying the variable indicator. One or more operating parameters of the IPC are calibrated based on information about the surgical tool stored within the IPC.

In aspects according to the present disclosure, the variable indicator is a resistive indicator. In other aspects according to the present disclosure, the resistive indicator is integrated within a removeable shaft of a surgical cutting device. In yet other aspects according to the present disclosure, the resistive indicator is aligned between two bearings that support the removeable shaft, each bearing residing in registration with a corresponding lead connected to the IPC.

In aspects according to the present disclosure, the variable indicator is a resistive coating disposed atop a removeable shaft of a surgical cutting device. In other aspects according to the present disclosure, the resistive coating is aligned between two bearings that support the removeable shaft, each bearing residing in registration with a corresponding lead connected to the IPC. In still other aspects according to the present disclosure, the resistive coating is configured to wear during use of the surgical tool effectively changing the electrical resistance of the surgical tool over time.

In aspects according to the present disclosure, the integrated power console monitors the condition of the surgical tool as a function of the wear of the resistive coating over time. In other aspects according to the present disclosure, the IPC associates a thickness of the resistive coating with a condition of the surgical tool and wherein, when the thickness reaches a threshold condition, the integrated power console recommends replacement.

Provided in accordance with aspects of the present disclosure is a method for identifying a surgical tool for use with a surgical device which includes reading, with a power console, e.g., an integrated power console, a variable indicator located within a removably engageable component of a surgical tool while a surgical device is attached to the integrated power console via one or more leads, the integrated power console: identifying the surgical tool based on quantifying the variable indicator; identifying a set of operating parameters for the surgical tool from a collection of operating parameters stored within the integrated power console; and calibrating the integrated power console according to the identified set of operating parameters.

### BRIEF DESCRIPTION OF THE DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of a surgical system in accordance with the present disclosure including an integrated power console (IPC) and a powered surgical cutting device;

FIGS. 2A and 2B illustrate side views of a powered surgical cutting device for use with the system of FIG. 1 including a variable indicator integrally disposed within a shaft of a surgical tool for use with the surgical cutting device in accordance with one embodiment of the present disclosure;

FIG. 2C illustrates an enlarged, internal side view of the powered surgical cutting device of FIG. 1 including the variable indicator integrally disposed within the shaft of a removeable tool for use with the surgical cutting device, and in alignment between two support bearings in accordance with one embodiment of the present disclosure;

FIGS. 3A-3C illustrate various views of a shaft of a surgical tool for use with a powered surgical cutting device for use with the system of FIG. 1 and having a resistive coating disposed about an outer periphery of the shaft;

FIGS. 4A-4C illustrate cross-sectional views of the shaft of the surgical tool with the resistive coating of FIG. 3A showing how a thickness of the coating wears during use over time;

FIG. 4D illustrates a cross-sectional view of the shaft of the surgical tool with the resistive coating of FIG. 3A showing a layered resistive coating atop the shaft; and

FIG. 5 illustrates a front view of a surgical saw for use with the surgical cutting device and the system of FIG. 1 including a variable indicator integrally disposed within the surgical saw in accordance with another embodiment of the present disclosure.

### DETAILED DESCRIPTION

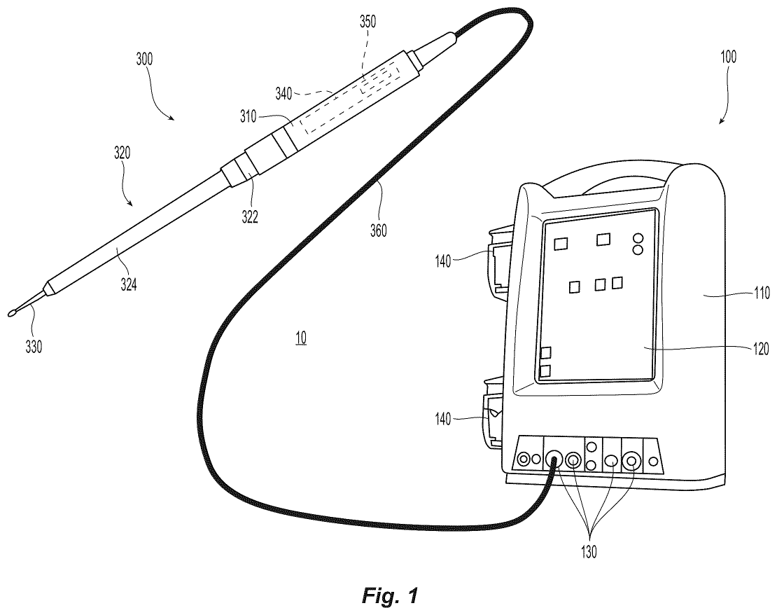

Turning to FIG. 1, a surgical system 10 provided in accordance with the present disclosure includes a console 100 and one or more surgical cutting devices 300. Console 100 may include an outer housing 110 enclosing the internal operable components of console 100, a touch screen graphical user interface (GUI) 120 to receive user input and display information to the user, a plurality of device ports 130, one or more fluid pumps 140, and/or other suitable features. One or more controllers including one or more processors and associated memory(s) are disposed within outer housing 110 and function to provide power and control signals to devices connected to console 100; to process user inputs, feedback data, and other data received at console 100; and to control the one or more fluid pumps 140. Suitable hardware and drive mechanisms as part of or in addition to controller may be disposed within outer housing 110 to perform the various functions of console 100 and may include, for example, one or more central processing units (CPU's) and/or microcontroller units (MCU's), power generating and control hardware and corresponding firmware/software stored thereon, sensor circuitry, motors, pump drivers, pump controllers, etc. It is envisioned that one or more aspects of the controller 100 (including one or more processors and associated memory(s)) may be operably associated with, removably associated with or contained within the handpiece or surgical device 300.

The one or more surgical cutting devices 300 may define any suitable configurations for use in performing various different surgical tasks, for use in various different procedures, etc. One example of a suitable surgical cutting device, surgical cutting device 300, generally includes a handle 310, a shaft assembly 320 extending distally from handle 310 (releasably or integrally connected thereto), a cutting tool 330 extending distally from shaft assembly 320 (releasably or integrally connected thereto), a motor 340 disposed within handle 310 and operably coupled to cutting tool 330 to drive rotation and/or reciprocation of cutting tool 330 relative to shaft assembly 320 to cut tissue, and a cord 360 to connect motor 340 to console 100 to enable console 100 to power and control motor 340, thereby controlling cutting tool 330. In aspects, shaft assembly 320 includes a rotation collar 322 that is rotatable relative to handle 310 to advance or retract (depending upon the direction of rotation of rotation collar 322) an outer sleeve 324 of shaft assembly 320 relative to cutting tool 330 to expose more or less of cutting tool 330 at the distal end of outer sleeve 324. Motor 340 may be an electric motor, pneumatic motor, ultrasonic transducer, or other suitable motor configured to drive cutting tool 330 to rotate and/or reciprocate for cutting tissue. Console 100 is configured to drive and control motor 340 such as, for example, a speed, torque, etc. output by motor 340. In aspects, surgical cutting device 300 may include additional features such as, for example, hand control(s), navigation, articulation, etc.

Cutting tool 330 may define any suitable configuration and may be integrated with surgical cutting device 300 or removable therefrom. Various different rotational cutting tools (not explicitly shown) may be configured for releasable attachment with surgical cutting device 300. In aspects, rotational cutting tools are releasably engageable with shaft assembly 320 (which, in turn, may be releasably or integrally connected to handle 310). Alternatively, rotational cutting tools 332 may be integral with corresponding shaft assemblies 320 that are, in turn, releasably engageable with handle 310. In either configuration, surgical cutting device 300 is thus capable of being interchangeably customized with a particular rotational cutting tool, depending upon a particular purpose. Reciprocating cutting tools and/or cutting tools configured for both rotation and reciprocation are also contemplated.

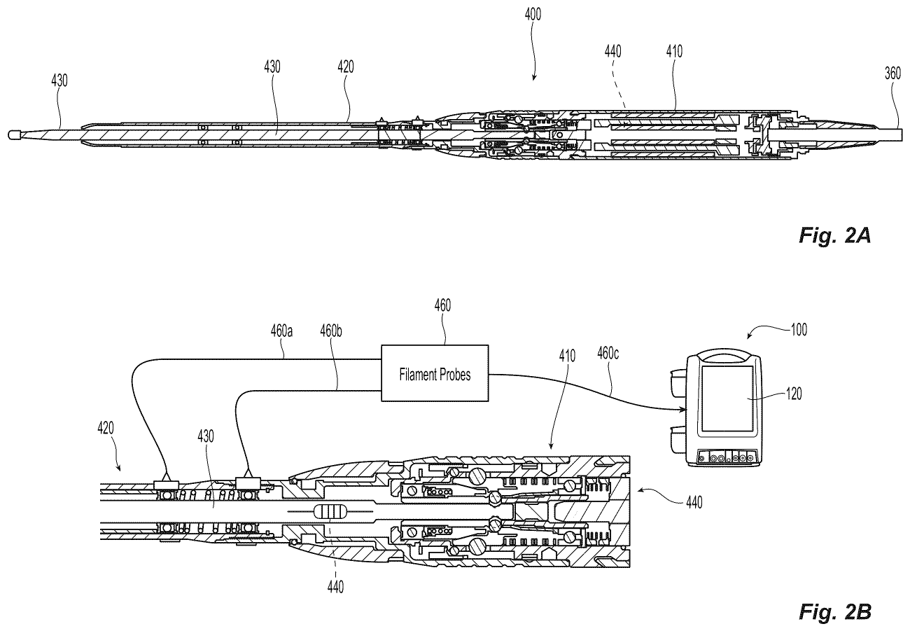

FIGS. 2A and 2B show another embodiment of a surgical cutting device 400 in accordance with the present disclosure including a handle 410, a shaft assembly 420 extending distally from handle 410 (releasably or integrally connected thereto), a cutting tool 430 extending distally from shaft assembly 420 (releasably or integrally connected thereto), a motor 440 disposed within handle 410 and operably coupled to cutting tool 430 to drive rotation and/or reciprocation of cutting tool 430 relative to shaft assembly 420 to cut tissue, and a cord 360 to connect motor 440 to console 100 to enable console 100 to power and control motor 440, thereby controlling cutting tool 430. The details relating to the shaft assembly 420, motor assembly 440 and console 100 are similar to the embodiments described above and, as such, are not described again for the purposes of brevity.

As described above, the surgical cutting device 400 is configured to operably and releasably engage surgical tool 430 which can include a variety of different types of surgical tools used for many different surgical purposes. As can be appreciated, surgical tools 430 may vary in shape, size, type, purpose or be specifically designed for use with particular surgical procedures. Moreover, surgical tools 430 may also vary based on operating parameters or be designed for use at a particular speed (RPM) and operating pressures. As such, each surgical tool 430 of surgical cutting device 400 includes a variable indicator 450 integrally disposed within shaft assembly 420 and configured for authentication prior to or after operative engagement with surgical device 400. For the purposes herein, variable indicator 450 can be any type of indicator that may be identifiable and quantifiable so that two or more components may be later distinguished, e.g., resistance, magnetic, inductance, capacitance, radioactive, etc. The term resistance indicator 450 is used below.

Visible indicia (not shown) may also be included on the shaft assembly 420 of each surgical tool 430 to identify the surgical tool 430 to the surgical personnel but the surgical tool 430 is also identifiable or readable by a probe, e.g., filament probe 460, associated with the IPC 100, thereby eliminating the need for the surgical personnel to manually program or authenticate the surgical tool 430 once engaged with the surgical device 400. It is envisioned that the IPC 100 may confirm accurate selection of the surgical tool 430, but the identification of the surgical tool 430 and calibration of the IPC 100 according to the specific operating parameters of the surgical tool 430 are automated once the surgical tool 430 is identified and confirmed.

One or more leads 460a, 460b are configured to measure the resistive indicator 450 in the shaft assembly 420 when engaged within handle 410 and the probe 460 is connected to the IPC 100 via a lead 460c. The resistance is measured on the GUI 120 associated with the IPC 100 and the tool 430 is identified and then the IPC 100 is calibrated accordingly. The outer surface of the surgical tool 430 may be marked with the tool's 430 known resistance based on standard resistance color code tables such as a Standard EIA Cold Code Table.

As can be appreciated, this avoids surgical personnel incorrectly programming operating parameters of a particular surgical tool 430 into the IPC, shortens operating time especially when utilizing multiple surgical tools 430, prevents re-use or reprocessing of surgical tools 430 (e.g., the IPC 100 may be configured to alert the surgical personnel prior to use if a surgical tool 430 has been previously used and/or reprocessed), tracks the individual surgical tool's usage and determine wear or expected life expectancy of a surgical tool 430 and recommends replacement if needed, i.e., logs a surgical tool's 430 usage or tool data (e.g., speed or other usage data), and authenticates manufacturer data.

FIG. 2C shows another embodiment of a surgical tool 430′ of surgical cutting device 400 which includes a resistive indicator 450′ integrally disposed within shaft assembly 420′ and configured for authentication prior to or after operative engagement with surgical device 400. The leads 460a, 460b are configured to measure the resistive indicator 450′ in the shaft assembly 420′ when engaged, i.e., when the resistive indicator 450′ is disposed between bearings 425a′ and 425b′ at a distal end of the handle 410′ and while the probe 460 is connected to the IPC 100 via a lead 460c. The resistance is measured on the GUI 120 associated with the IPC 100 and the surgical tool 430′ is identified and the IPC is then calibrated accordingly. Filament probes (not shown) may be configured to contact the shaft assembly 420′ or the shaft assembly 420′ may be spring-loaded to contact the resistance indicator 450′.

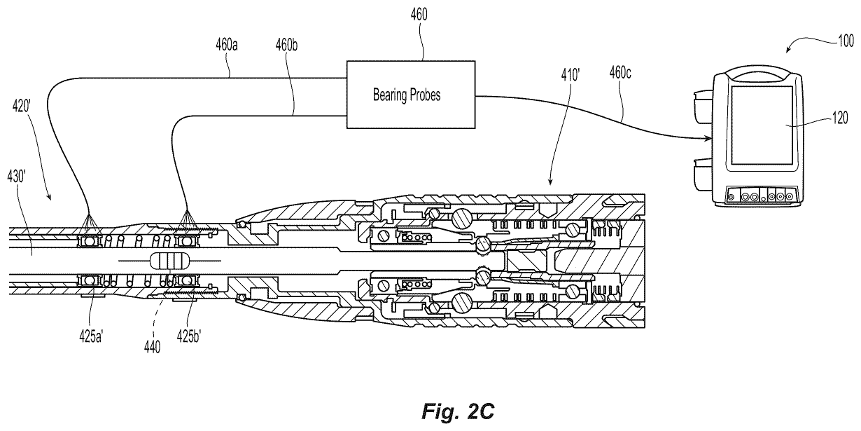

Turning now to FIGS. 3A-3C, as mentioned above, the surgical cutting device 400 is configured to operably and releasably engage a variety of different types of surgical tools used for many different surgical purposes. Surgical tools may vary in shape, size, type, purpose or be specifically designed for use with particular surgical procedures. As mentioned above, an internal resistive indicator 450 may be integrally associated with the shaft assembly 420. An electrical coating 550 may also be applied to an outer peripheral surface of the shaft assembly 520 of each surgical tool 530 to identify individual types of surgical tools 530 in a similar fashion by type, size, shape, purpose, operating parameter, etc. As such, each surgical tool 530 of surgical cutting device 400 includes an electrical coating 550 coated on the outer surface of the shaft assembly 520 and configured for authentication prior to or after operative engagement with surgical device 400. Visible indicia (not shown) may also be included on the shaft assembly 520 of each surgical tool 530 to identify the surgical tool 530 to the surgical personnel.

Upon engagement within the surgical device 400, electrical coating 550 is identifiable or readable by a probe, e.g., filament probe 460 (See FIG. 2B), associated with the IPC 100, thereby eliminating the need for the surgical personnel to manually program or authenticate the surgical tool 530 once engaged with the surgical device 400. Once the surgical tool 530 is identified, the IPC 100 calibrates itself accordingly and prior to activation confirms accurate selection of the surgical tool 530 with surgical personnel prior to activation. Similar to FIG. 2B, one or more leads (460a, 460b-See FIG. 2B) measure the electrical properties of the electrical coating 550 to both identify and monitor the surgical tool 530 during use. As can be appreciated, this eliminates surgical personnel from incorrectly programming in the operating parameters of a particular surgical tool 530, shortens operating time especially when utilizing multiple surgical tools 530, and enables the IPC 100 to authenticate a manufacturer.

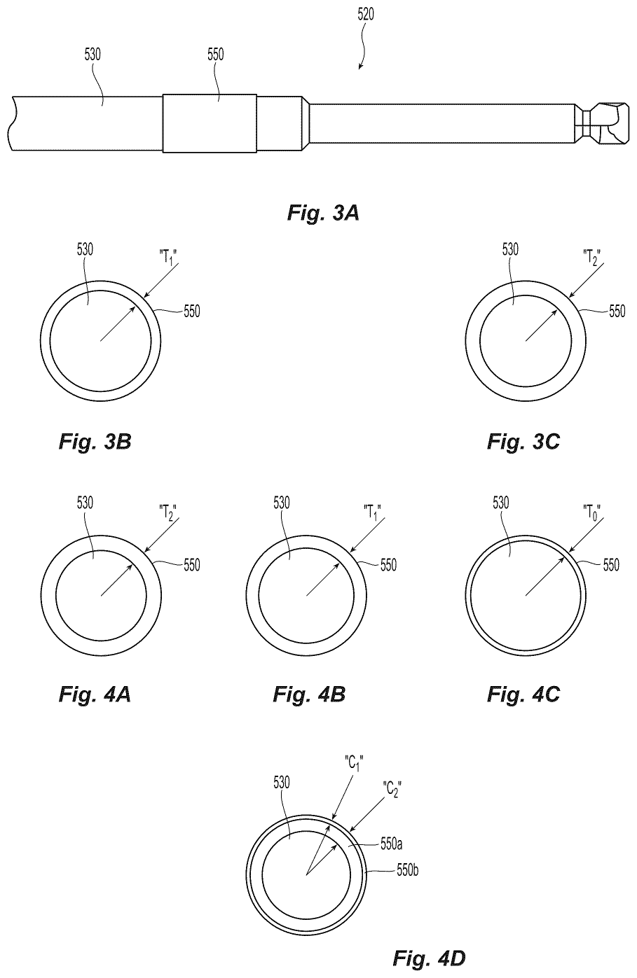

Moreover and with specific reference to FIGS. 4A and 4B, the IPC 100 may be configured to prevent re-use or reprocessing of surgical tools 530 by tracking an individual surgical tool's usage via the overall wear of the electrical coating 550 as it corresponds to the expected life expectancy of a surgical tool 530 and recommend replacement, i.e., log a surgical tool's 530 usage or tool data (e.g., speed or other usage data). For example, over time, the outer electrical coating 550 may initially come from the manufacturer and be identified as a particular surgical tool 530 which has certain electrical properties including an electrical coating 550 with a thickness of “T2”. During use, the electrical coating 550 may wear (e.g., as the shaft 520 rides atop two bearings, e.g., bearings 425a′, 425b′-FIG. 2C) and, as such, the electrical properties would change as the thickness changes from “T2” to “T1” to “TO”. When the thickness of electrical coating 550 reaches “TO”, the IPC 100 may signal the user to deactivate the surgical device 400 and replace the surgical tool 530 with another similar tool at that time. Or, alternatively, the surgical tool 530 may simply not be usable in a new procedure (when a subsequent identification and calibration is performed).

In embodiments, multiple electrical coatings 550a, 550b may be employed having varying electrical properties (different quantifying criteria) such that during initial use (or shortly thereafter) a dramatic change in the electrical properties takes effect as the first or outer layer wears (FIG. 4D). For example, the outer electrical coating C2 550b may include a higher electrical resistance than the inner electrical coating C1 550a, e.g., by an order or magnitude to clearly differentiate the change and allow the inner electrical coating C1 to vary by surgical tool 530 as intended. As such, each surgical tool 530 includes the same outer electrical coating C2 550b that immediately wears upon use (or within a short period of time) and a different inner electrical coating C1 550a (as described above) to determine the identification of the specific surgical tool 530. The IPC 100 during calibration expects to read the combination resistance of both electrical coatings C1 550a plus C2 550b or the IPC 100 identifies an error, e.g., the loaded surgical tool 530 was previously used or reprocessed. Different quantifying criteria may include: an order of magnitude of resistance or different electrical parameter (e.g., a thin microcircuit outer layer which provides a separate electrical signal to the IPC that acts as a manufacturing seal that ruptures during initial use).

In embodiments, the IPC 100 may be configured to monitor the use of the surgical tool 530 as a function of the wear of the resistive coating 550 (or 550a, 550b) over time.

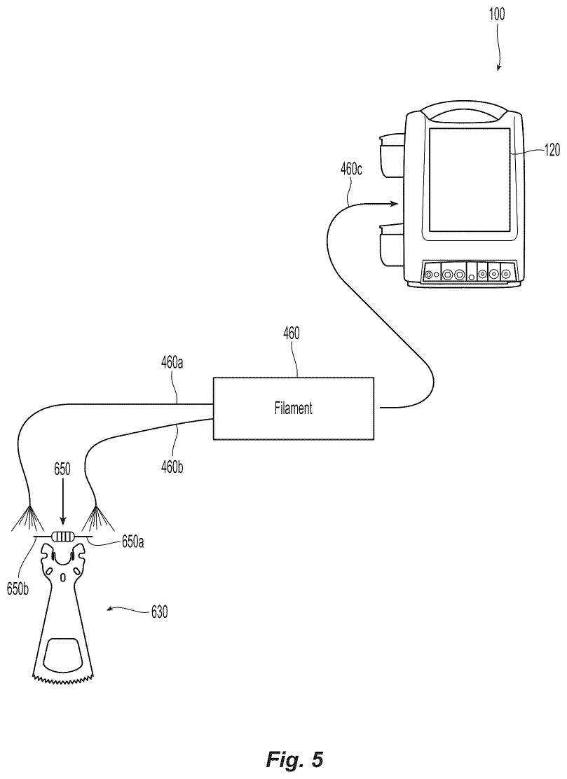

Turning now to FIG. 5, another embodiment of a resistive indicator for use with a surgical device, e.g., surgical cutting device 400, is shown and is generally identified as resistive indicator 650. Resistive indicator 650 may be configured in a similar fashion as described above with respect to FIGS. 2A-4D. In other words, resistive indicator 650 may be integrally associated with a surgical saw 630 as described with respect to FIGS. 2A-2C or coated thereon in a similar fashion as described above with respect to FIGS. 3A-4D.

Prior to engagement, upon engagement or during use, surgical saw 630 is identifiable or readable by a probe, e.g., filament probe 460 (See FIG. 2B), associated with the IPC 100, thereby eliminating the need for the surgical personnel to manually program or authenticate the surgical saw 630 once engaged with the surgical device 400. Once the surgical saw 630 is identified, the IPC 100 calibrates itself accordingly and prior to activation may confirm accurate selection of the surgical saw 630 with surgical personnel prior to activation. Similar to FIGS. 2B and 3A-3C above, leads 460a, 460b measure the electrical properties of the resistive indicator 650 (or electrical coating) to both identify and monitor the surgical saw 630 during use. This eliminates programming errors, enables manufacturing authentication, and prevents re-use.

While several aspects of the present disclosure have been shown in the drawings, it is not intended that the present disclosure be limited thereto, as it is intended that the present disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

What is claimed is:

1. A method for identifying a surgical tool for use with a surgical device, comprising:
integrating a variable indicator within a removably engageable component of a surgical tool of a surgical device;
reading the variable indicator with a power console upon engagement of the surgical tool with the surgical device via one or more leads connected to the power console and identifying the surgical tool based on quantifying the variable indicator; and
calibrating the power console according to the operating parameters of the surgical tool stored within the power console.

2. The method for identifying a surgical tool for use with a surgical device according to claim 1, wherein the variable indicator is a resistive indicator.

3. The method for identifying a surgical tool for use with a surgical device according to claim 2, further comprising integrating the resistive indicator within a removeable shaft of a surgical cutting device.

4. The method for identifying a surgical tool for use with a surgical device according to claim 3, wherein the resistive indicator is aligned between two bearings that support the removeable shaft, each bearing residing in registration with a corresponding lead connected to the power console.

5. The method for identifying a surgical tool for use with a surgical device according to claim 1, wherein the variable indicator is a resistive coating.

6. The method for identifying a surgical tool for use with a surgical device according to claim 5, further comprising applying the resistive coating atop a removeable shaft of a surgical cutting device.

7. The method for identifying a surgical tool for use with a surgical device according to claim 6, further comprising aligning the resistive coating between two bearings that support the removeable shaft, each bearing residing in registration with a corresponding lead connected to the power console.

8. The method for identifying a surgical tool for use with a surgical device according to claim 7, wherein the resistive coating is configured to wear during use of the surgical tool effectively changing the electrical resistance of the surgical tool over time.

9. The method for identifying a surgical tool for use with a surgical device according to claim 8, further comprising monitoring the condition of the surgical tool as a function of the wear of the resistive coating over time.

10. The method for identifying a surgical tool for use with a surgical device according to claim 8, further comprising layering the resistive coating to prevent reuse, wherein each layer includes a different quantifier.

11. The method for identifying a surgical tool for use with a surgical device according to claim 9, further comprising:
removing the surgical tool from the surgical device when the power console associates a thickness of the resistive coating to a condition of the surgical tool recommending replacement; and
replacing the surgical tool.

12. A system for identifying a surgical tool for use with a surgical device, comprising:
a selectively removable tool of a surgical device, the selectively removeable tool including an integrated variable indicator disposed therein; and
one or more leads configured to align with the removable tool upon engagement of the tool with the surgical device, the one or more leads connected to a power console configured to read the variable indicator and identify the surgical tool based on quantifying the variable indicator, wherein one or more operating parameters of the power console are calibrated based on information about the surgical tool stored within the power console.

13. The system for identifying a surgical tool for use with a surgical device according to claim 12, wherein the variable indicator is a resistive indicator.

14. The system for identifying a surgical tool for use with a surgical device according to claim 13, wherein the resistive indicator is integrated within a removeable shaft of a surgical cutting device.

15. The system for identifying a surgical tool for use with a surgical device according to claim 14, wherein the resistive indicator is aligned between two bearings that support the removeable shaft, each bearing residing in registration with a corresponding lead connected to the power console.

16. The system for identifying a surgical tool for use with a surgical device according to claim 12, wherein the variable indicator is a resistive coating disposed atop a removeable shaft of a surgical cutting device.

17. The system for identifying a surgical tool for use with a surgical device according to claim 16, wherein the resistive coating is aligned between two bearings that support the removeable shaft, each bearing residing in registration with a corresponding lead connected to the power console.

18. The system for identifying a surgical tool for use with a surgical device according to claim 17, wherein the resistive coating is configured to wear during use of the surgical tool effectively changing the electrical resistance of the surgical tool over time.

19. The system for identifying a surgical tool for use with a surgical device according to claim 18, wherein the power console monitors the condition of the surgical tool as a function of the wear of the resistive coating over time.

20. A method for identifying a surgical tool for use with a surgical device, comprising:
reading, with a power console, a variable indicator located within a removably engageable component of a surgical tool while a surgical device is attached to the power console via one or more leads, the power console:
identifying the surgical tool based on quantifying the variable indicator,
identifying a set of operating parameters for the surgical tool from a collection of operating parameters stored within the power console; and
calibrating the power console according to the identified set of operating parameters.
